(19) United States
(12) Patent Application Publication
(10) Pub. No.: US 2025/0348795 A1
(43) Pub. Date: Nov. 13, 2025

(54) PLASMA CENTER QUEUE MANAGEMENT SYSTEM

(71) Applicant: Haemonetics Corporation, Boston, MA (US)

(72) Inventor: Melvin Tan, Boston, MA (US)

(21) Appl. No.: 19/202,533

(22) Filed: May 8, 2025

Related U.S. Application Data
(60) Provisional application No. 63/644,210, filed on May 8, 2024.

(51) Int. Cl. G06Q 10/02; G16H 40/20

(52) U.S. Cl. CPC G06Q 10/02; G16H 40/20

(57) ABSTRACT

A method of managing plasma donation throughput of a plasma donor center includes receiving a request message indicating a request from a donor to donate plasma at the plasma donor center. Plasma donor center data is received from two or more of a plasma donor management system, a plasma center queue management system, or a donor plasma donation mobile application. The wait time is determined within the plasma donor center as a function of plasma center data received from at least two of a plasma donor management system, a plasma center queue management system, or a donor plasma donation mobile application. A reservation message is forwarded to a blood establishment computer system regarding the queue position of the donor, the reservation, or both the donor and the reservation, and a confirmation message is forwarded toward including the determined wait time and preferred arrival time for the donor.

### CROSS REFERENCE TO RELATED APPLICATIONS

This Application claims priority from U.S. Provisional Patent Application No. 63/644,210, filed May 8, 2024, the contents of which are incorporated by reference herein in its entirety as if fully set forth.

### FIELD

Illustrative embodiments of the invention generally relate to blood processing and, more particularly, various embodiments of the invention relate to managing plasma donations in a plasma center.

### BACKGROUND

Plasma donation is based on human donors in which whole blood is drawn from a donor and processed into individual blood components, such as plasma. A person who intends to donate plasma generally visits a plasma center where plasma center staff will examine and process the donor in accordance with the plasma center procedures until such time that the donor is deemed suitable or unsuitable for donating plasma.

Plasma centers use donor software to manage the entire donor process, including determining the suitability of a donor to donate plasma. Among other things, the donor software often tracks one or more of the processing, status, and movement of a donor as the donor is processed at each stage of the visit.

The donor also may use a donor mobile application to signal their intent to donate plasma on a certain day and time. Favorably, this provides the plasma center staff foresight into upcoming donor arrival pattern. A networked apheresis device collects plasma from the donor. Accordingly, the apheresis device typically includes an embedded software program that tracks the phlebotomy procedure being performed by the apheresis device, including procedure information such as start time, stop time, stage, status, cycle number, cycle time, volume, weight, errors, and other related information.

Moreover, plasma centers are busy facilities with donor traffic that varies throughout the day. A donor often times their arrival at the plasma center to minimize wait time, or time in a physical queue if the plasma center is busy. If the queue wait times in the plasma center are too long, the donor may become disincentivized from donating at that specific day or time. Because of individual donor preferences and/or limitations to donate at a specific plasma center for a specific day or time, some donors may be unable to avoid a donation visit at the plasma center during a busy time.

### SUMMARY OF VARIOUS EMBODIMENTS

In accordance with one embodiment, a method of managing plasma donation throughput of a plasma donor center. The method includes receiving a request message via a wide area network or a local area network indicating a request from a donor to donate plasma at the plasma donor center, the plasma donor center having a plurality of apheresis devices in communication with a blood establishment computer system (BECS) via the wide area network or the local area network; receiving recent plasma donor center data from two or more of a) a plasma donor management system, b) a plasma center queue management system, or c) a donor plasma donation mobile application, the recent plasma donor center data comprising one or more of current apheresis device usage, location information relating to the donor via the donor plasma donation mobile application, and apheresis device stage information; determining the wait time within the plasma donor center as a function of plasma center data received from at least two of a) a plasma donor management system, b) a plasma center queue management system, or c) a donor plasma donation mobile application; positioning donor information in a determined queue position of a virtual queue to form a donor reservation; forwarding a reservation message to the BECS regarding the donor and determined queue position of the donor, the reservation, or both the donor and the reservation, the reservation message being forwarded via the wide area network or the local area network; and forwarding a confirmation message toward the donor via the network, the confirmation message including the determined wait time and preferred arrival time for the donor.

In some embodiments, the method further includes determining donor eligibility before forwarding the confirmation message.

In some embodiments, the method further includes receiving an eligibility message from the BECS confirming donor eligibility.

In some embodiments, the plasma center data comprises the number of donors in the plasma system and the current stage of each donor.

In some embodiments, the method further includes using geolocation to determine the number of donors in the plasma center, further wherein the recent plasma center data comprises the determined number of donors in the plasma center.

In some embodiments, the recent plasma center data comprises the number of donors scheduled to arrive at the plasma center during a prescribed time frame, the number of donors determined based on queue spots secured broken down by the scheduled donor arrival time or time slots.

In some embodiments, the recent plasma center data comprises the number of apheresis devices in use in the plasma center at a given time.

In some embodiments, the wait time within the plasma center is determined as a function of plasma donor center data received from each of a) the plasma donor management systems, b) the plasma center queue management system, c) donor plasma donation mobile application, or d) at least one apheresis device.

In some embodiments, a geolocation application detects the donor and forwards ID information across the network indicating the presence of the donor in a prescribed vicinity of the plasma donor center, the method further comprising forwarding a request message to the donor, in response to receipt of the ID information, requesting a donation from the donor and/or the wait time.

In accordance with one embodiment, a plasma donation system for managing plasma donation throughput of a plasma donor center includes an interface in communication with a wide area network or a local area network, the interface configured to receive a request message via the wide area network and/or a local area network indicating a request from a donor to donate plasma at the plasma donor center, the plasma donor center having a plurality of apheresis devices, the interface being in communication with a blood establishment computer system (BECS) via the wide area network or the local area network, the recent plasma donor center data comprising one or more of current apheresis device usage in the plasma donor center, location information relating to the donor via the donor plasma donation mobile application, and the stage of usage of the plurality of apheresis devices; a timer operatively coupled with the interface, the timer configured to determine the wait time within the plasma donor center as a function of plasma center data received, via the interface, from at least two of a) a plasma donor management system, b) a plasma center queue management system, or c) a donor plasma donation mobile application; and a queue scheduler configured to position donor information in a determined queue position of a virtual queue to form a donor reservation, the queue scheduler configured to form a reservation message for forwarding to the BECS regarding the determined queue position of the donor, the donor reservation, or both the determined queue position and the donor reservation. The interface is configured to forward, via the wide area network or the local area network, the reservation message. The interface is also configured to forward a confirmation message toward the donor via the wide area network or local area network, the confirmation message including the determined wait time and preferred arrival time for the donor.

In some embodiments, the plasma donation system further includes the plasma donor management system determining donor eligibility before forwarding the confirmation message.

In some embodiments, the plasma donation system further includes the plasma donor management system receiving an eligibility message from the BECS confirming donor eligibility.

In some embodiments, the plasma center data comprises the number of donors in the plasma system and the current stage of each donor.

In some embodiments, the plasma donation system further includes the plasma center queue management system using geolocation to determine the number of donors in the plasma center, further wherein the recent plasma center data comprises the determined number of donors in the plasma center.

In some embodiments, the recent plasma center data comprises the number of donors scheduled to arrive at the plasma center during a prescribed time frame, the number of donors determined based on queue spots secured broken down by the scheduled donor arrival time or time slots.

In some embodiments, the recent plasma center data comprises the number of apheresis devices in use in the plasma center at a given time.

In some embodiments, the wait time within the plasma center is determined as a 10 function of plasma donor center data received from each of a) the plasma donor management systems, b) the plasma center queue management system, c) donor plasma donation mobile application, or d) at least one apheresis device.

In some embodiments, the plasma donation system further includes a geolocation application detects the donor and forwards ID information across the network indicating the presence of the donor in a prescribed vicinity of the plasma donor center, the method further comprising forwarding a request message to the donor, in response to receipt of the ID information, requesting a donation from the donor and/or the wait time.

Illustrative embodiments are implemented as a computer program product having a computer usable medium with computer readable program code thereon. The computer readable code may be read and utilized by a computer system in accordance with conventional processes.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1 schematically shows one implementation of a plasma donation system as it interacts within a network in accordance with illustrative embodiments.

FIG. 2 schematically shows a perspective view of a blood/plasma processing system in accordance with illustrative embodiments.

FIG. 3 schematically shows a plan view of the blood/plasma processing system of FIG. 2.

FIG. 4 schematically shows an example of a disposable set installed within the blood/plasma processing system of FIG. 2 in accordance with illustrative embodiments.

FIG. 5 schematically shows a plasma donation system in accordance with illustrative embodiments of the invention.

FIG. 6 shows a method of managing scheduling and service of plasma donor center donors in accordance with illustrative embodiments.

FIG. 7 is an example block diagram of a device in accordance with illustrative embodiments.

### DESCRIPTION OF ILLUSTRATIVE EMBODIMENTS

Illustrative embodiments more efficiently and effectively manage scheduling in a unique environment—within a plasma center. To that end, a plasma donation system determines donor wait times after arrival at the plasma center as a function of data from one or more of a) a donor management system, b) a queue management system, and c) a donor mobile application. Using geolocation technology, the plasma donation system also can locate and request a donation from a potential donor in a specified geographical region, and strategically position that or another donor in a virtual plasma donation queue before or after arrival at the plasma center. Details of illustrative embodiments are discussed below.

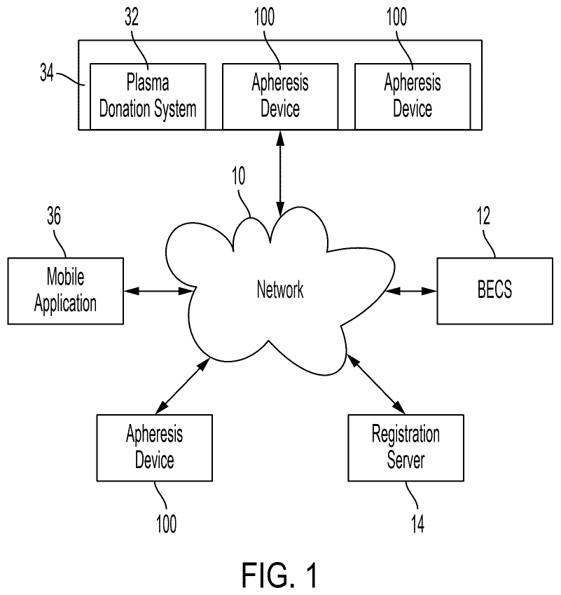

FIG. 1 schematically shows one implementation of a plasma donation system 32 as it interacts with other connected network networked devices in accordance with illustrative embodiments. As shown in this embodiment, the plasma donation system 32 is within a single physical location/site (e.g., a permanent or mobile plasma center 34) with a plurality of on-premises devices apheresis devices 100 (discussed in detail below). The plasma donation system 32 also may manage one or more off-premises devices (e.g., other apheresis devices 100) via a network 10. As shown, the network 10 may include a wide area network (e.g., the Internet), or some other network configuration (e.g., a local area network) communicating a plurality of devices.

The apheresis devices 100 communicate with a registration server 14 in accordance with illustrative embodiments. In this example, three apheresis devices 100 operating within the same functional or business unit are controlled and coordinated by a set of one or more remote device registration servers 14. Among other things, the functional/business entity operating the apheresis devices 100 may be a single plasma center 34. As known by those in the art and discussed in greater detail below, the registration server 14 manages data flow and registration with the apheresis devices 100 and communication with other electronic management systems, such as a remotely located blood establishment computer system (“BECS 12”).

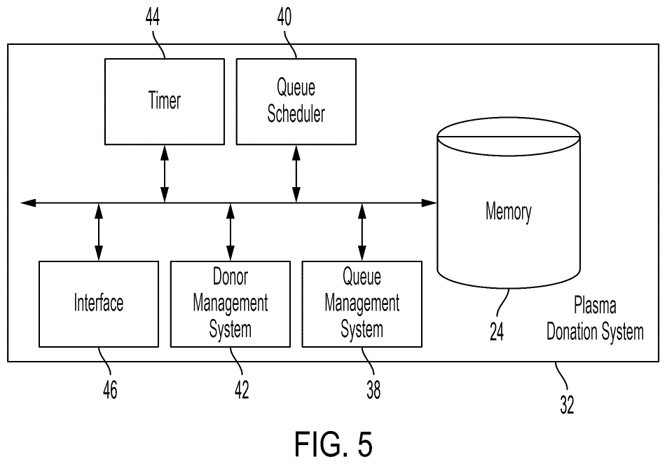

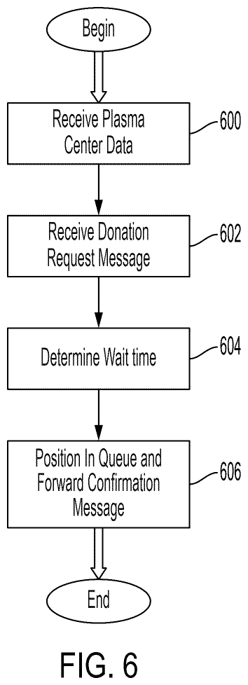

The person who may or will donate plasma (referred to generally as the “donor”) may have an associated software application to interact with the plasma donation system 32. For example, the donor may have a mobile telephone or smartphone with a mobile application that interacts with the plasma donation system 32. This mobile application may be used by the donor to augment the donation experience, such as completing required donor questionnaires prior to arrival at the plasma center 34. It also may have geolocation software or other functionality to communicate with the plasma center 34—e.g., a user interface to schedule an appointment. FIGS. 5 and 6 show more detail of this interaction.

Indeed, it should be noted that like other figures, FIG. 1 is a simplified figure intended to demonstrate the environment of various embodiments. Those skilled in the art may add further functionality or reduce some functionality shown in that figure. Accordingly, FIG. 1 is illustrative and not intended to limit various embodiments.

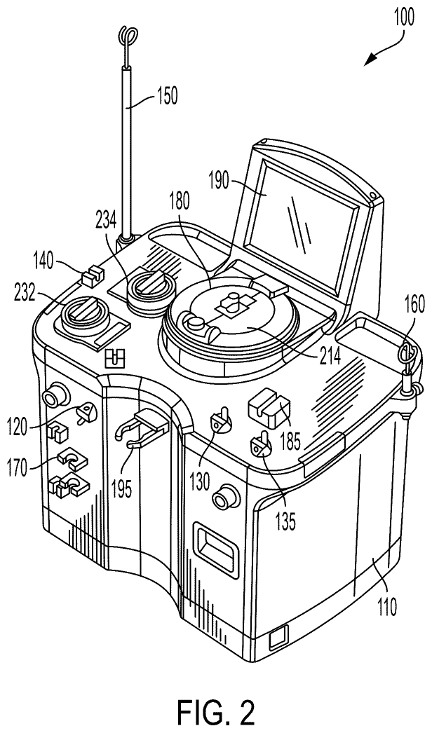

FIGS. 2-4 detail some components making up the network 10 and associated network devices as configured in illustrative embodiments and described above with regard to FIG. 1.

Generally speaking, apheresis devices 100 are medical devices designed to selectively remove specific components from a person's blood while returning the remaining blood components back to the individual. As discussed below in greater detail with regard to FIGS. 2-4, the apheresis device 100 has several primary components. First, it incorporates a system for accessing the individual's blood, often through the insertion of intravenous lines or catheters. The device then uses various methods, such as centrifugation or filtration, to separate the blood components based on their physical or chemical properties. The desired components are collected into specialized containers or bags for further processing or use. Throughout the process, apheresis devices 100 incorporate monitoring systems and control mechanisms to ensure accuracy and safety. These systems may include sensors, pumps, and software interfaces that regulate flow rates, volumes, and other parameters.

Safety features are a crucial aspect of apheresis devices 100. They can include alarms for pressure or flow irregularities, air detection systems, and safety interlocks to protect the donor, patient, and the operator. A pheresis procedures have a wide range of therapeutic applications, including manufacturing into therapies, collecting blood components for transfusion, removing excess or abnormal substances from the blood, and treating specific medical conditions. Plateletpheresis, plasmapheresis, and leukapheresis are examples of therapeutic apheresis procedures.

In medical settings, such as blood or plasma centers, hospitals, and specialized clinics, as well in mobile settings, apheresis devices 100 are employed by trained professionals to perform these procedures. The devices supply the necessary technology and control to efficiently and safely separate blood components. Their use is critical for addressing various therapeutic needs and ensuring the well-being of patients undergoing apheresis procedures.

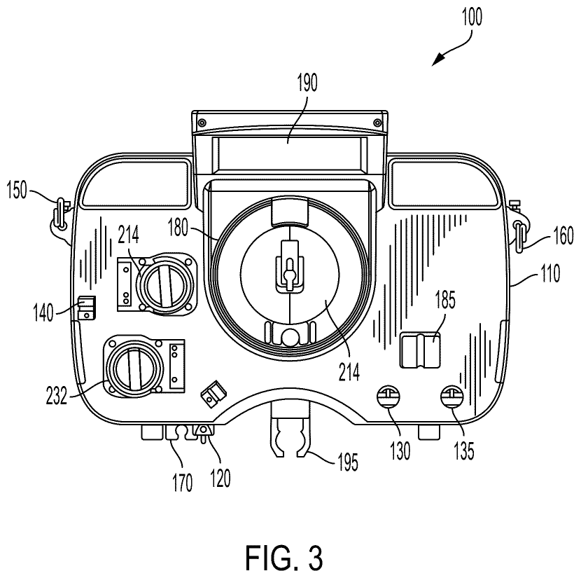

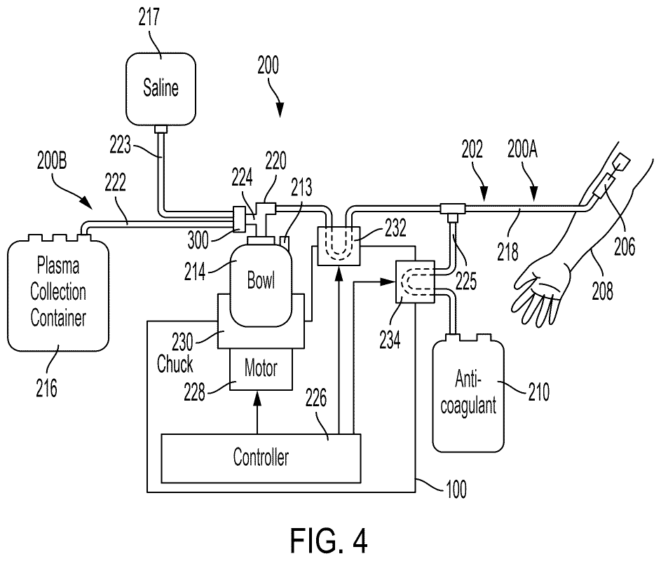

FIG. 2 schematically shows a perspective view of a blood processing system that may be used with illustrative embodiments. FIG. 3 schematically shows a plan view of the blood processing system of FIG. 2. As shown, the blood processing/apheresis device/system 100 includes a cabinet 110 that houses the main components of the system 100 (e.g., the non-disposable components). Within the cabinet 110, the system 100 may include a first/blood pump 232 that draws whole blood from a subject, and a second/anticoagulant pump 234 that pumps anticoagulant through the system 100 and into the drawn whole blood. Additionally, the system 100 may include a number of valves that may be opened and/or closed to control the fluid flow through the system 100. For example, the system 100 may include a donor valve 120 that may open and close to selectively prevent and allow fluid flow through a donor line 218 (e.g., an inlet line shown in FIG. 4), and a plasma valve 130 that selectively prevents and allows fluid flow through an outlet/plasma line 222 (FIG. 4). Some embodiments may also include a saline valve 135 that selectively prevents and allows saline to flow through a saline line 223.

To facilitate the connection and installation of a disposable set and to support the corresponding fluid containers, the system 100 may include an anticoagulant pole 150 on which the anticoagulant solution container 210 (FIG. 4) may be hung, and a saline pole 160 on which a saline solution container 217 (FIG. 4) may be hung (e.g., if the procedure being performed requires the use of saline). Additionally, in some applications, it may be necessary and/or desirable to filter the whole blood drawn from the subject for processing. To that end, the system 100 may include blood filter holder 170 in which the blood filter (located on the disposable set) may be placed.

As discussed in greater detail below, apheresis systems 100 in accordance with illustrative embodiments withdraw whole blood from a subject through a venous access device 206 (FIG. 4) using the blood pump 232. As the system 100 withdraws the whole blood from the subject, the whole blood enters a blood component separation device 214, such as a Latham type centrifuge (other type of separation chambers and devices may be used, such as, without limitation, an integral blow-molded centrifuge bowl, as described in U.S. Pat. Nos. 4,983,158 and 4,943,273). The blood component separation device 214 separates the whole blood into its constituent components (e.g., red blood cells, white blood cell, plasma, and platelets). Accordingly, to facilitate operation of the separation device 214, the system 100 may also include a well 180 in which the separation device 214 may be placed and in which the separation device 214 rotates (e.g., to generate the centrifugal forces required to separate the whole blood).

To allow the user/technician to monitor the system operation and control/set the various parameters of the procedure, the system 100 may include a user interface 190 (e.g., a touch screen device) that displays the operation parameters, any alarm messages, and buttons which the user/technician may depress to control the various parameters. Additional components of the blood processing system 100 are discussed in greater detail below (e.g., in relation to the system operation).

FIG. 4 schematically shows, as a block diagram, the blood processing system 100 and a disposable collection set 200 (with an inlet disposable set 200A and an outlet disposable set 200B) that may be loaded onto/into the blood processing system 100, in accordance with the illustrative embodiments. The collection set 200 includes a venous access device 206 (e.g., a phlebotomy needle) for withdrawing blood from a donor's arm 208, a container of anti-coagulant 210, a centrifugation bowl 214 (e.g., a blood component separation device), a saline container 217, and a final plasma collection bag 216. The blood/inlet line 218 couples the venous access device 206 to an inlet port 220 of the bowl 214, the plasma/outlet line 222 couples an outlet port 224 of the bowl 214 to the plasma collection bag 216, and a saline line 223 connects the outlet port 224 of the bowl 214 to the saline container 217. An anticoagulant line 225 connects the anti-coagulant container 210 to the inlet line 218. In addition to the components mentioned above and as shown in FIG. 4, the blood processing system 100 includes a controller 226, a motor 228, and a centrifuge chuck 230. The controller 226 is operably coupled to the two pumps 232 and 234, and to the motor 228, which, in turn, drives the chuck 230. The controller 226 may be operably coupled to and in communication with the user interface 190.

In operation, the disposable collection set 200 (e.g., the inlet disposable set 200A and the outlet disposable set 200B) may be loaded onto/into the blood processing system 100 prior to blood processing. In particular, the blood/inlet line 218 is routed through the blood/first pump 232 and the anticoagulant line 225 from the anti-coagulant container 210 is routed through the anticoagulant/second pump 234. The centrifugation bowl 214 may then be securely loaded into the chuck 230. After the bowl 214 is secured in place, the technician may install the outlet disposable set 200B. For example, the technician may connect a bowl connector 300 to the outlet 224 of the bowl 214, install the plasma container 216 into the weight senor 195, run the saline line 223 through valve 135, and run the plasma/outlet line 222 through valve 130 and the line sensor 185. After the disposable set 200 is installed and the anticoagulant and saline containers 210/217 are connected, the system 100 is ready to begin blood processing.

A pheresis devices 100 are utilized with donors or patients in a controlled and monitored environment, ensuring the safety and well-being of the individual undergoing the procedure. The process typically begins with careful donor or patient preparation and assessment to determine their eligibility and suitability for apheresis.

Before the procedure, the donor's or patient's vital signs, medical history, and relevant laboratory tests are reviewed to ensure that they meet the specific criteria for apheresis. The donor or patient is informed about the procedure, its purpose, and any potential risks or side effects. Informed consent is obtained to ensure that the donor or patient understands the nature of the procedure and provides their agreement to proceed.

After the donor or patient is prepared, an appropriate blood access point is established to facilitate the collection and return of blood. This may involve the insertion of one or more intravenous lines or catheters, depending on the specific requirements of the apheresis procedure. The access points are carefully chosen to minimize discomfort and ensure adequate blood flow during the process.

Next, the apheresis device 100 is set up and configured based on the prescribed parameters for the procedure. This includes programming the device with the desired settings, such as flow rates, separation protocols, and collection volumes, which are tailored to the individual patient's needs.

During the procedure, the apheresis device 100 carefully extracts blood from the donor or patient through the established blood access point. The blood flows through the device, where, as noted with regard to FIGS. 2-4, it undergoes the separation process, either by centrifugation or filtration. The targeted blood component, such as platelets, plasma (in embodiments), or white blood cells, is selectively collected while the remaining blood components are returned to the patient.

Throughout the procedure, the patient's vital signs, including blood pressure, heart rate, and oxygen saturation, are closely monitored to ensure their safety and well-being. The apheresis device's monitoring systems continuously assess critical parameters, such as flow rates, pressures, and component levels, allowing operators to make real-time adjustments if necessary.

Once the desired amount of the targeted component is collected or the prescribed procedure time is reached, the apheresis device 100 completes the process. The donor's or patient's blood access points are carefully removed, and appropriate post-procedure care and monitoring are provided to ensure their comfort and recovery.

The collected blood components often undergo further processing, testing, and preparation as required for their intended therapeutic use. This can involve additional steps such as component labeling, manufacturing, storage, and compatibility testing to ensure their safety and effectiveness when administered to patients.

As noted, the apheresis devices 100 communicate and are controlled in the network 10 via the noted registration server 14. Unlike a typical network server, the registration server 14 has a special role in the apheresis process. Specifically, the registration server 14 in various embodiments acts as a centralized system that facilitates the registration, monitoring, and/or connectivity of apheresis devices 100 from the same locations and/or remote locations. As such, the registration server 14 streamlines the registration and connectivity process for apheresis devices 100 located at various static and/or mobile sites. It further provides a standardized mechanism for registering and identifying individual devices, allowing users (e.g., providers) to have a comprehensive view of the devices within their network 10.

Operation of the apheresis device 100 may be performed in either disconnected mode or connected mode. In the disconnected mode, the apheresis device 100 does not receive or send data to any network enabled computer software system or device.

Instead, data collected by the apheresis device 100 remains on the device itself. In connected mode, however, the apheresis device 100 can receive and send data to network enabled computer software systems or devices. The flow of data is bi-directional, meaning it can both send and receive data from external systems.

As noted above, apheresis devices 100 from a single or related unit (e.g., a plasma center 34) can be physically located in different facilities or clinical settings (e.g., see FIG. 1). The registration server 14 therefore acts as a central hub that enables remote management and monitoring of these devices. In addition, the registration server 14 may track assets and manage device activation in case devices 100 are lost or stolen. Registration may involve receiving device-specific information, such as model numbers, activation codes, license information, unique identifiers, and location details. The registration process ensures that each device is properly activated, licensed, authorized, and integrated into the software system, facilitating seamless communication and control.

Importantly, the registration server 14 enables remote connectivity and communication with registered apheresis devices 100. It provides a secure and reliable means for remote operators or administrators to access and manage the devices. Through this connectivity, operators can monitor device status, review operational parameters, and perform remote troubleshooting or maintenance tasks as needed. The registration server 14 thus can act as the bridge that facilitates the exchange of data and commands between the remote operators and the apheresis devices 100.

The registration server 14 can be implemented in different ways, depending on the specific setup and requirements of the apheresis devices 100 and the organization. For example, the registration server 14 can be located on the same site as the apheresis devices 100, remotely hosted in an off premises (e.g., in a secure data center), or a combination of both. On-site implementations can facilitate direct and immediate access to these and other devices from the local network 10. Operators or administrators can register and manage the devices within the same physical location, facilitating efficient communication and control. In certain scenarios, a combination of both local and remote configurations may be implemented. This hybrid approach may involve a local registration server 14 at each site where apheresis devices 100 are present, while also connecting to a central remote registration server 14 for higher-level management and coordination. In some embodiments, this setup allows for a balance between local control and centralized oversight. Other embodiments facilitate centralized oversight of the various devices 100 (e.g., a single entity having oversight of many devices 100 across may locations).

The choice of deployment configuration depends on factors, such as the number and distribution of apheresis devices 100, licensing and activation requirements, the organization's IT infrastructure, connectivity requirements, and security considerations. One goal is to ensure seamless connectivity, effective device management, and reliable access to the apheresis devices 100, regardless of whether the registration server 14 is on-site, remote, or a combination of both.

The apheresis device 100 preferably is configured to operate in the connected mode and to automatically connect with the registration server 14. When the apheresis device 100 connects to the Internet (whether through the noted network of FIG. 1 or some other connection), it will automatically register itself with the registration server 14 by sending data about itself, including specific identification data such as serial identifier, location, timestamps, component versions, or any relevant device data such as procedure data, user, or location specific information. After receipt, the registration server 14 preferably processes, stores, and provides a response to the apheresis device 100. This response may include a simple confirmation of device registration, activation codes, licenses, delivery of update files, programming instructions, or new messages received from an external network-enabled system, such as the noted BECS 12 or another networked device. A BECS 12 is shown here as one exemplary implementation.

As known by those in the art, the BECS 12 typically plays a significant role in the optimization of blood banking and plasma collection operations. Designed specifically for blood establishments, these specialized systems offer comprehensive functionalities to manage the collection, testing, processing, storage, and distribution of blood and blood products. When appropriately configured, the BECS 12 can often ensure the safety, traceability, and efficiency of the blood and blood products supply chain.

An important aspect of the BECS 12 in some embodiments involves donor management. This device favorably facilitates the registration and screening of donors, capturing crucial demographic information and medical history. It tracks the lifecycle of a donor's engagement, including donation records, deferrals, and notifications for repeat donations. With robust donor management capabilities, blood establishments can maintain accurate and up-to-date donor data, ensuring the eligibility and safety of the donated blood products.

Another important structure within the BECS 12 involves component management. This functionality allows blood establishments (e.g., a plasma center 34) to track the processing and manufacturing of various blood components, such as red blood cells, platelets, plasma, and cryoprecipitate. By closely monitoring the inventory and location of these components, the BECS 12 enables efficient allocation and usage, minimizing waste and ensuring optimal stock rotation. Additionally, component management ensures proper labeling and tracking, enabling swift retrieval and traceability when needed.

Quality control and testing are integral parts of any plasma center operation, and the BECS 12 streamlines these processes. The system typically integrates laboratory testing results, including compatibility testing, infectious disease screening, and component quality assessment. Test results are securely stored within the BECS 12, facilitating quick access and retrieval when needed. Moreover, the system manages the inventory of test kits and reagents, ensuring their availability and tracking expiration dates. By automating testing and quality control procedures, the BECS 12 enhances accuracy, efficiency, and regulatory compliance.

Reporting and regulatory compliance also form an important aspect of plasma operations. To that end, the BECS 12 generates a wide range of reports, including blood and plasma inventory, donor statistics, adverse events, and performance indicators. These reports not only provide valuable insights for internal management, while also aiding in complying with national and international standards and guidelines. The BECS 12 thus serves as a reliable tool for blood and plasma collection establishments to demonstrate adherence to regulatory requirements and continuously monitor and improve their operations. Some of these functions can cooperate with the apheresis device 100 and registration server 14 to appropriately and effectively complete blood donation and processing.

In the embodiment shown, the registration server 14 acts as a middleware type software element (“middleware”) in the network 10 between the BECS 12 and the apheresis device 100. To that end, the registration server 14 preferably has a middleware engine acting as a bridge, facilitating communication and data exchange between the apheresis devices 100 and the BECS 12. As known by those in the art, middleware engines (also known as “interface engines”) are configured to handle data integration and interoperability between different systems that may use different communication protocols or have incompatible interfaces. They act as a mediator, translating the data formats, protocols, and commands used by the BECS 12 and the apheresis device 100, enabling seamless communication.

Some embodiments may use one or more of the following steps when employing middleware or interface engines:
Integration Configuration: The middleware or interface engine is configured to understand the data formats and communication protocols used by both the BECS 12 and the apheresis device 100. This configuration allows the middleware to act as a translator between the two systems.
Data Translation: The middleware translates the data from the BECS 12 into a format that the apheresis device 100 can understand, and vice versa. This involves mapping the data elements, converting data structures, and ensuring compatibility between the systems.
Protocol Conversion: If the BECS 12 and the apheresis device 100 use different communication protocols, the middleware converts the data transmission to align with the protocols supported by both systems. This ensures that the data can be successfully transmitted and received by each system.
Command Routing: The middleware receives commands from the BECS 12 and routes them to the apheresis device 100. It ensures that the commands are properly formatted and translated to the appropriate commands understood by the apheresis device 100.

Similarly, the middleware receives data and status updates from the apheresis device 100 and forwards them to the BECS 12.

By utilizing a middleware or interface engine, plasma centers 34 can achieve interoperability between their BECS 12 and apheresis devices 100 that may have different communication capabilities or protocols. This approach enables seamless data transfer, command execution, and status monitoring between the two systems.

Middleware or interface engines often adhere to industry standards for healthcare data exchange, such as Health Level Seven (HL7) or Integrating the Healthcare Enterprise (IHE). These standards ensure compatibility and interoperability across different systems within the healthcare domain, facilitating smooth integration and communication between the BECS 12 and the apheresis device 100.

Alternative embodiments may use other approaches for interconnecting the elements of the network 10. For example, in some cases, the BECS 12 may have built-in capabilities to directly integrate with compatible apheresis devices 100. This integration may be achieved through standardized communication protocols or interfaces. The BECS 12 sends commands and receives data directly from the apheresis device 100, allowing for seamless data exchange and control. Other embodiments may have device-specific interfaces. For example, apheresis devices 100 often have their own software interfaces or application programming interfaces (APIs) that enable communication with external systems like the BECS 12. These interfaces provide a standardized way for the BECS 12 to interact with the apheresis device 100, allowing for data transfer, command execution, and status updates. In either case, some embodiments provide the desired plug-and-play capability for the apheresis devices 100.

Accordingly, discussion of the specific interconnections, as well as some of the devices above (e.g., the BECS 12) are illustrative and not intended to limit various embodiments of the invention.

FIG. 5 schematically shows selected components making up the plasma donation system 32 (“plasma system 32”) of FIG. 1 in accordance with illustrative embodiments. Each of these components is operatively connected by any conventional interconnect mechanism—Figure 5 simply shows a bus communicating each the components. Those skilled in the art should understand that this generalized representation can be modified to include other conventional direct or indirect connections. Accordingly, discussion of a bus is not intended to limit various embodiments.

Indeed, FIG. 5 only schematically shows selected components/parts of the plasma system 32. Those skilled in the art should understand that each of these components can be implemented in a variety of conventional manners, such as by using hardware, software, or a combination of hardware and software, across one or more other functional components. For example, the timer 44 (to be discussed) may be implemented using a plurality of microprocessors executing firmware. As another example, the timer 44 may be implemented using one or more application specific integrated circuits (i.e., “ASICs”) and related software, or a combination of ASICs, discrete electronic components (e.g., transistors), and microprocessors. Accordingly, the representation of the timer 44 and other components in a single box of FIG. 5 is for simplicity purposes only. In fact, in some embodiments, the timer 44 or other parts shown in FIG. 5 can be distributed across a plurality of different machines—not necessarily within the same housing or chassis.

It should be reiterated that the representation of FIG. 5 is a significantly simplified representation of the plasma system 32. Those skilled in the art should understand that such a device has many other physical and functional components, such as central processing units, other communication processing modules, and short-term memory. Accordingly, this discussion is not intended to suggest that FIG. 5 represents all of the elements of the plasma system 32.

As shown, the plasma system 32 has memory 24 (e.g., long-term memory) for storing information, such as data from various data generating devices, donor information, wait times, and a virtual queue of donors. This virtual queue sets the order for donors to donate their plasma. Accordingly, to manage this queue, the plasma system 32 also has a) a queue management system 38 to position and/or slot a donor for arrival to the plasma center 34 and to manage the donor's status in the queue upon arrival (e.g., move the donor's position in the queue if late, cancel if too late, etc.), and b) a queue scheduler 40 configured to cause the queue management system 38 to:
1) position donor information in a determined queue position of a virtual queue, and
2) 2) forward a confirmation message toward the donor (e.g., to the donor application) via the network 10.

Indeed, those skilled in the art may combine the functionality of the queue management system 38 and queue scheduler 40 into a single logical entity and/or physical entity.

The plasma system 32 also has a donor management system 42 configured to manage the entire donor process. For example, the donor management system 42 may determine the suitability of a donor to donate plasma, and track processing, status, and movement of a donor as the donor is processed at each stage of the plasma donation process.

Illustrative embodiments communicate with the donor to share and receive any of a variety of types of information. Among other things, the plasma system 32 may determine an approximate wait time at the plasma center 34 at one or more times of the day. To that end, the plasma system 32 may forward a message to the donor's mobile application 36 indicating the wait time—e.g., the amount of time that will elapse from the time the donor arrives at the plasma center 34 until the donor is seated or positioned at a phlebotomy bed on the donation floor.

To effectuate that, the plasma system 32 has a timer 44 configured to determine donor wait times within the plasma center 34. As discussed below, this wait time preferably is determined as a function of center data received from at least two of several different well known plasma donation systems: 1) the donor management system 42, 2) the queue management system 38, and 3) the donor mobile application. Some embodiments also may determine this wait time as a function of information received from the apheresis devices 100 in the plasma center 34. FIG. 6 discusses some embodiments of how the timer 44 makes these determinations. Finally, the plasma system 32 has an interface 46 to transmit and/or receive information from an external device, such as one of the apheresis devices 100, the mobile application 36, the BECS 12, and/or the registration server 14 of FIG. 1.

FIG. 6 shows a method of managing scheduling and servicing plasma center donors in accordance with illustrative embodiments. It should be noted that this process is substantially simplified from a longer process that normally would be used. Accordingly, this process may have additional steps that those skilled in the art likely would use. In addition, some of the steps may be performed in a different order than that shown, or at the same time. Those skilled in the art therefore can modify the process as appropriate. Moreover, as noted above and below, the specific processes and structures in each step are exemplary. As such, those skilled in the art can use other processes and/or structures to accomplish the goal of one or more steps, depending upon the application and other constraints. Accordingly, discussion of specific processes and structures is not intended to limit all embodiments.

As noted above, the process of FIG. 6 begins at step 600, which receives plasma center data from any one, two, or three of 1) the donor management system 42, 2) the queue management system 38, and 3) the mobile application. In addition to or instead of one or more of these three data sources, some embodiments also may use information from one or more of the apheresis devices 100 managed by the plasma center 34. As discussed in detail below, this plasma center data may include any of a variety of information relating to the donor, plasma center components, activity at the plasma center 34, etc., and is used to determine the wait time. This information can be continually accumulated and stored in memory 24, and may be controlled by the timer 44, queue scheduler 40, or some controller (not shown) in the plasma system 32. Alternatively, or in addition, this data can be received on demand, such as automatically upon receipt of a plasma donation request.

Accordingly, after receiving a donation request message from a potential donor (step 602), the timer 44 (automatically or upon operator request) determines the approximate wait time for that donor at one or more specified arrival times in the plasma center 34 (step 604). That donation request can be generated from any of a variety of means. For example, a geolocation application associated with the donor application may detect that the donor is within a certain prescribed proximity of the plasma center 34 (e.g., within one mile from the plasma center 34), responsively prompting the donor management system 42 to send an automated invitation to donate plasma. That message may include approximate times for the donor to arrive and wait times (i.e., after step 604). The donor may reply with a message indicating their willingness to donate. Other embodiments, however, may not rely on the automatic geolocation data and simply may receive a request from the donor to donate plasma (e.g., via the donor application after not donating for some specified time frame).

Next, the timer 44 determines the wait time at step 604. As suggested above, the timer 44 makes that determination as a function of plasma system data received from any of a variety of sources, such as those discussed above. The timer 44 may use a formula, calculation (e.g., linear, multiple, polynomial, exponential or other regression calculations), artificial intelligence, or other way to use that system data to determine the wait time. When using a formula, one skilled in the art may weigh certain data more than other data.

As noted, an artificial intelligence system (“AI System”) may make informed assumptions and predictions based on the input data. For example, once equipped with the necessary data inputs/training, the AI system may employ several techniques to calculate wait times effectively. Note that other techniques, such as those using a formula, calculation, etc. also may use some of these techniques as appropriate. Those techniques may include any one or combinations of one or more of the following:
Historical Analysis: The AI system conducts an in-depth analysis of historical data to identify patterns and trends. This historical perspective allows it to recognize peak appointment times, such as Mondays or Fridays, which tend to result in longer wait times. Consequently, the system can make more accurate predictions.
Queue Management: The AI system continually monitors the queue's status. It estimates wait times by considering variables such as the number of donors ahead, the pace of service, and historical data.
Location-Based Predictions: The AI system adapts its calculations based on the dynamics of the location. For example, if the plasma center 34 is situated in a densely populated urban area with heavy traffic, the system factors in potential delays attributable to congestion.
Staffing Optimization: The AI system may take into account the availability and proficiency of staff members, including their schedules, to provide more accurate predictions.
Real-time Adjustments: To maintain prediction accuracy, the AI system continuously incorporates real-time updates. It dynamically adjusts its estimates as new information emerges, accommodating unexpected delays or early completions.
User Preferences: The AI system may account for user preferences, allowing individuals to specify their willingness to wait or their preference for shorter wait times. This information enables the system to tailor predictions to user expectations.
Feedback Loop: AI systems can integrate user feedback into their decision-making processes. Deviations between predicted and actual wait times can be analyzed to enhance future predictions, fostering continuous improvement.

Below are some examples of information that both the AI system and/or formulaic approach can use. Illustrative embodiments may use any one or combinations of one or more of the data types below:
The number of donors that are in the plasma center 34, broken down by and/or based on the current stage of each donor.
Data from a plurality of donor mobile applications 36 of a plurality of donors can be used to calculate the number of donors that are in the plasma center 34. This may be completed in a number of manners, such as by using the mobile application's geolocation features.
Data from the plurality of donor mobile applications 36 indicating the number of donors planning to arrive at the plasma center based on the donor's completion of questionnaires and/or messages indicating their arrival may be used.
Data from the plurality of networked apheresis devices 100 that are in use, and/or data from all of the networked apheresis devices 100 regardless of whether they are in use can be used. Data from the plurality of networked apheresis devices 100 may be used to determine the current real-time status of devices (e.g., to determine which are in use).
Data from the queue management system 38 and/or queue scheduler 40 can be used to calculate the number of donors that are planning to arrive at the plasma center based on spots that were secured in the queue,
Data from the queue system can be used to calculate the number of donors that are planning to arrive at the plasma center based on spots that were secured in the queue, broken down by the arrival time or time slots.

In illustrative embodiments, the estimated wait time for a donor is made available through an application programming interface (API). Moreover, the estimated wait time for a donor may be displayed on the donor mobile application 36. Those skilled in the art can forward the estimated wait time to a plurality of donors using an allowed channel, such as email, text messaging, instant messaging, mobile application push notification, mobile application, in-app notification, or an automated telephone call. The estimated wait time for a donor also may be communicated to a plurality of plasma center staff using a configured channel, such as email, text messaging, instant messaging, automated telephone call, donor system notifications, a donor system report, or a donor system screen/dashboard. The estimated wait time for a donor preferably is used by the queue management system 38 to determine spots and/or time slots that are available for a donor to secure in the plasma center donor queue.

Accordingly, the process continues to step 606, in which the queue scheduler 40/queue management system 38 positions the donor in the queue and forwards a confirmation message to the donor with the wait time. In one embodiment, the donor communicates with the plasma system 32 via their mobile application 36 (or other software) to secure their position in the queue from any remote location. After receiving the donor request to the join the queue (e.g., from any remote or local donor location), the queue management system 38 receives an identification number for the donor, processes the request for a position on the queue for that intended donor center, and assigns the donor at least one secured position in the queue. The queue management system 38 and/or queue scheduler 40 then designates an optional arrival time or time range for the queue position and returns a confirmation message to the donor that their position in the queue has been secured. This message also may include the wait time at one or more times.

After the donor has secured a position in the queue, the donor may check-in upon arrival at the plasma center 34 in any of a variety of manners. For example, the donor may use a computing device connected to the queue management system 38 to indicate the donor's arrival and jump/place that donor to their designated position in the queue. Some embodiments may automatically check-in the donor merely upon detecting the donor's arrival at the plasma center 34 (e.g., using geolocation software on the donor's mobile device). As such, in this and other cases, the donor does not necessarily have to interact overtly with plasma center personnel or devices to be assigned a spot in the queue.

In some embodiments, if the donor does not arrive or check-in within the designated time or time range for the intended plasma center 34, the donor's secured queue position is automatically extended for a predetermined time period. The donor may or may not be notified to about the extension. Alternatively, when the donor has secured a position in the queue for the intended plasma center 34, but the donor does not arrive or check-in within a designated time or time range, the donor's secured queue position is automatically released, and the donor may be notified about this change.

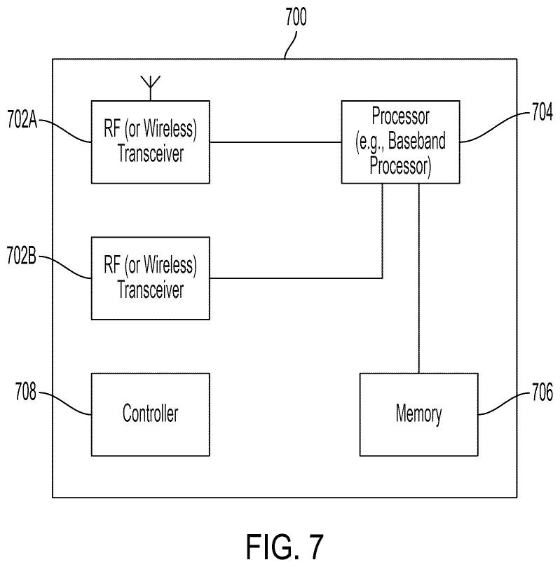

FIG. 7 is an example block diagram of a device 700 in accordance with illustrative embodiments. In various embodiments, the device 700 may be any one of the devices depicted in FIG. 1 and may be a wired or wireless device. For purposes of example, device 700 is depicted as a wireless device in FIG. 7.

In various embodiments, the device 700 may include, for example, one or more (e.g., two as shown in FIG. 7) RF (radio frequency) or wireless transceivers 702A, 702B, where each wireless transceiver includes a transmitter to transmit signals and a receiver to receive signals. The device 700 also includes a processor or control unit/entity (controller) 704 to execute instructions or software and control transmission and receptions of signals, and a memory 706 to store data and/or instructions.

Processor 704 may also make decisions or determinations, generate frames, packets or messages for transmission, decode received frames or messages for further processing, and other tasks or functions described herein. Processor 704, which may be a baseband processor, for example, may generate messages, packets, frames or other signals for transmission via wireless transceiver 702 (702A or 702B). Processor 704 may control transmission of signals or messages over a wireless network, and may control the reception of signals or messages, etc., via a wireless network (e.g., after being down-converted by wireless transceiver 702, for example). Processor 704 may be programmable and capable of executing software or other instructions stored in memory or on other computer media to perform the various tasks and functions described above, such as one or more of the tasks or methods described above. Processor 704 may be (or may include), for example, hardware, programmable logic, a programmable processor that executes software or firmware, and/or any combination of these. Using other terminology, processor 704 and transceiver 702 together may be considered as a wireless transmitter/receiver system, for example.

In addition, referring to FIG. 7, a controller (or processor) 708 may execute software and instructions, and may provide overall control for the station 700, and may provide control for other systems not shown in FIG. 7, such as controlling input/output devices (e.g., display, keypad), and/or may execute software for one or more applications that may be provided on wireless device 700, such as, for example, an email program, audio/video applications, a word processor, a Voice over IP application, or other application or software.

In addition, a storage medium may be provided that includes stored instructions, which when executed by a controller or processor may result in the processor 704, or other controller or processor, performing one or more of the functions or tasks described above.

According to another example embodiment, RF or wireless transceiver(s) 702A/702B may receive signals or data and/or transmit or send signals or data. Processor 704 (and possibly transceivers 702A/702B) may control the RF or wireless transceiver 702A or 702B to receive, send, broadcast or transmit signals or data.

Example embodiments are provided or described for each of the example methods, including: An apparatus (e.g., 700, FIG. 7) including means (e.g., processor 704, RF transceivers 702A and/or 702B, and/or memory 706, in FIG. 7) for carrying out any of the methods; a non-transitory computer-readable storage medium (e.g., memory 706, FIG. 7) comprising instructions stored thereon that, when executed by at least one processor (processor 704, FIG. 7), are configured to cause a computing system (e.g., 700, FIG. 7) to perform any of the example methods; and an apparatus (e.g., 700, FIG. 7) including at least one processor (e.g., processor 704, FIG. 7), and at least one memory (e.g., memory 706, FIG. 7) including computer program code, the at least one memory (706) and the computer program code configured to, with the at least one processor (704), cause the apparatus (e.g., 700) at least to perform any of the example methods.

Various embodiments of the invention may be implemented at least in part in any conventional computer programming language. For example, some embodiments may be implemented in a procedural programming language (e.g., “C”), or in an object-oriented programming language (e.g., “C++”). Other embodiments of the invention may be implemented as a pre-configured, stand-alone hardware element and/or as preprogrammed hardware elements (e.g., application specific integrated circuits, FPGAS, and digital signal processors), or other related components.

In an alternative embodiment, the disclosed apparatus and methods (e.g., see the various flow charts described above) may be implemented as a computer program product for use with a computer system. Such implementation may include a series of computer instructions fixed either on a tangible, non-transitory medium, such as a computer readable medium (e.g., a diskette, CD-ROM, ROM, solid state drive, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the invention may be implemented as a combination of both software (e.g., a computer program product) and hardware. Still other embodiments of the invention are implemented as entirely hardware, or entirely software.

The embodiments of the invention described above are intended to be merely exemplary; numerous variations and modifications will be apparent to those skilled in the art. Such variations and modifications are intended to be within the scope of the present invention as defined by any of the appended claims.

What is claimed is:

1. A method of managing plasma donation throughput of a plasma donor center, the method comprising:
receiving a request message via a wide area network or a local area network indicating a request from a donor to donate plasma at the plasma donor center, the plasma donor center having a plurality of apheresis devices in communication with a blood establishment computer system (BECS) via the wide area network or the local area network;
receiving recent plasma donor center data from two or more of a) a plasma donor management system, b) a plasma center queue management system, or c) a donor plasma donation mobile application,
the recent plasma donor center data comprising one or more of current apheresis device usage, location information relating to the donor via the donor plasma donation mobile application, and apheresis device stage information;
determining the wait time within the plasma donor center as a function of plasma center data received from at least two of a) a plasma donor management system, b) a plasma center queue management system, or c) a donor plasma donation mobile application;
positioning donor information in a determined queue position of a virtual queue to form a donor reservation;
forwarding a reservation message to the BECS regarding the donor and determined queue position of the donor, the reservation, or both the donor and the reservation, the reservation message being forwarded via the wide area network or the local area network; and
forwarding a confirmation message toward the donor via the network, the confirmation message including the determined wait time and preferred arrival time for the donor.

2. The method of claim 1 further comprising determining donor eligibility before forwarding the confirmation message.

3. The method of claim 2 further comprising receiving an eligibility message from the BECS confirming donor eligibility.

4. The method of claim 1 wherein the plasma center data comprises the number of donors in the plasma system and the current stage of each donor.

5. The method of claim 1 further comprising using geolocation to determine the number of donors in the plasma center, further wherein the recent plasma center data comprises the determined number of donors in the plasma center.

6. The method of claim 1 wherein the recent plasma center data comprises the number of donors scheduled to arrive at the plasma center during a prescribed time frame, the number of donors determined based on queue spots secured broken down by the scheduled donor arrival time or time slots.

7. The method of claim 1 wherein the recent plasma center data comprises the number of apheresis devices in use in the plasma center at a given time.

8. The method of claim 1 wherein the wait time within the plasma center is determined as a function of plasma donor center data received from each of a) the plasma donor management systems, b) the plasma center queue management system, c) donor plasma donation mobile application, or d) at least one apheresis device.

9. The method of claim 1 wherein a geolocation application detects the donor and forwards ID information across the network indicating the presence of the donor in a prescribed vicinity of the plasma donor center, the method further comprising forwarding a request message to the donor, in response to receipt of the ID information, requesting a donation from the donor and/or the wait time.

10. A plasma donation system for managing plasma donation throughput of a plasma donor center, the method comprising:
an interface in communication with a wide area network or a local area network, the interface configured to receive a request message via the wide area network and/or a local area network indicating a request from a donor to donate plasma at the plasma donor center, the plasma donor center having a plurality of apheresis devices, the interface being in communication with a blood establishment computer system (BECS) via the wide area network or the local area network, the recent plasma donor center data comprising one or more of current apheresis device usage in the plasma donor center, location information relating to the donor via the donor plasma donation mobile application, and the stage of usage of the plurality of apheresis devices;
a timer operatively coupled with the interface, the timer configured to determine the wait time within the plasma donor center as a function of plasma center data received, via the interface, from at least two of a) a plasma donor management system, b) a plasma center queue management system, or c) a donor plasma donation mobile application; and
a queue scheduler configured to position donor information in a determined queue position of a virtual queue to form a donor reservation, the queue scheduler configured to form a reservation message for forwarding to the BECS regarding the determined queue position of the donor, the donor reservation, or both the determined queue position and the donor reservation,
the interface configured to forward, via the wide area network or the local area network, the reservation message,
the interface also configured to forward a confirmation message toward the donor via the wide area network or local area network, the confirmation message including the determined wait time and preferred arrival time for the donor.

11. The plasma donation system of claim 10 further comprising the plasma donor management system determining donor eligibility before forwarding the confirmation message.

12. The plasma donation system of claim 11 further comprising the plasma donor management system receiving an eligibility message from the BECS confirming donor eligibility.

13. The plasma donation system of claim 10 wherein the plasma center data comprises the number of donors in the plasma system and the current stage of each donor.

14. The plasma donation system of claim 10 further comprising the plasma center queue management system using geolocation to determine the number of donors in the plasma center, further wherein the recent plasma center data comprises the determined number of donors in the plasma center.

15. The plasma donation system of claim 10 wherein the recent plasma center data comprises the number of donors scheduled to arrive at the plasma center during a prescribed time frame, the number of donors determined based on queue spots secured broken down by the scheduled donor arrival time or time slots.

16. The plasma donation system of claim 10 wherein the recent plasma center data comprises the number of apheresis devices in use in the plasma center at a given time.

17. The plasma donation system of claim 10 wherein the wait time within the plasma center is determined as a function of plasma donor center data received from each of a) the plasma donor management systems, b) the plasma center queue management system, c) donor plasma donation mobile application, or d) at least one apheresis device.

18. The plasma donation system of claim 10 wherein a geolocation application detects the donor and forwards ID information across the network indicating the presence of the donor in a prescribed vicinity of the plasma donor center, the method further comprising forwarding a request message to the donor, in response to receipt of the ID information, requesting a donation from the donor and/or the wait time.

19. A computer program product for use on a computer system for managing plasma donation throughput of a plasma donor center, the computer program product comprising a tangible, non-transient computer usable medium having computer readable program code thereon, the computer readable program code comprising:
program code for receiving a request message via a wide area network and/or a local area network indicating a request from a donor to donate plasma at the plasma donor center, the plasma donor center having a plurality of apheresis devices in communication with a blood establishment computer system (BECS) via the wide area network or the local area network;
program code for receiving recent plasma donor center data from two or more of a) a plasma donor management system, b) a plasma center queue management system, or c) a donor plasma donation mobile application,
the recent plasma donor center data comprising one or more of current apheresis device usage, location information relating to the donor via the donor plasma donation mobile application, and apheresis device stage information;
program code for determining the wait time within the plasma donor center as a function of plasma center data received from at least two of a) a plasma donor management system, b) a plasma center queue management system, or c) a donor plasma donation mobile application;
program code for positioning donor information in a determined queue position of a virtual queue to form a donor reservation;
program code for forwarding a reservation message to the BECS regarding the donor and determined queue position of the donor, the reservation, or both the donor and the reservation, the reservation message being forwarded via the wide area network or the local area network; and
program code for forwarding a confirmation message toward the donor via the network, the confirmation message including the determined wait time and preferred arrival time for the donor.

20. The computer program product of claim 19 wherein the plasma center data comprises the number of donors in the plasma system and the current stage of each donor.
